(19) United States
(12) Patent Application Publication
(10) Pub. No.: US 2026/0263547 A1
(43) Pub. Date: Sep. 10, 2026

(54) COMPOSITION FOR PREVENTING, AMELIORATING, OR TREATING ARTHRITIS, CONTAINING POTENTILLA CHINENSIS EXTRACT OR APIGENIN-7-O-GLUCURONIDE AS ACTIVE INGREDIENT

(71) Applicant: BELABELBIO INC., Gimhae-si, Gyeongsangnam-do (KR)

(72) Inventors: Byung Chan JUNG, Boseong-gun, Jeollanam-do (KR); Sun Hee LEE, Hwaseong-si, Gyeonggi-do (KR); Kyung-Tae LEE, Seoul (KR)

(21) Appl. No.: 19/167,797

(22) PCT Filed: Mar. 28, 2024
(86) PCT No.: PCT/KR2024/003968

§ 371 Date: Sep. 22, 2025

(30) Foreign Application Priority Data
Apr. 19, 2023 (KR) 10-2023-0051096
Mar. 27, 2024 (KR) 10-2024-0041923

(51) Int. Cl. A61K 36/73; A61K 31/7048; A61P 19/02; A61P 29/00

(52) U.S. Cl. CPC A61K 36/73; A61K 31/7048; A61P 19/02; A61P 29/00; A61K 2236/333

(57) ABSTRACT

Disclosed is a composition for the prevention, improvement, or treatment of arthritis, including an Extract of Potentilla chinensis or apigenin-7-O-glucuronide as an active ingredient. The present invention can inhibit the expression of MMP-1 and MMP-9, increase the synthesis of procollagen type II, alleviate inflammation, and suppress inflammatory pain, thereby preventing, improving, or treating arthritis.

### TECHNICAL FIELD

The present invention relates to a composition for the prevention, improvement, or treatment of arthritis.

### BACKGROUND ART

Degenerative arthritis is a disease in which inflammation and pain occur due to damage to bones, ligaments, and other joint structures caused by progressive damage or degenerative changes of the cartilage that protects the joint, and is the most common among inflammatory joint diseases. It is classified into primary or idiopathic arthritis, which occurs depending on factors such as age, sex, genetic factors, obesity, and specific joint sites without any particular organic cause, and secondary arthritis, which is caused by trauma, diseases, or deformities that may damage the joint cartilage.

Most patients suffer from primary arthritis, and it is known to affect about 10 to 15% of the global population. In addition, about 31% of the adult population in Korea exhibits symptoms, making it one of the representative degenerative diseases. The number of patients with arthritis continues to increase due to extended human life expectancy and population aging.

Degenerative arthritis is a disease caused by degenerative changes such as aging occurring in chondrocytes that constitute the joint, wherein the synthesis of extracellular matrix components of the joint, such as type-II collagen and proteoglycan, in the chondrocytes is inhibited, while inflammatory cytokines such as interleukin-1β and tumor necrosis factor-α are produced. As a result, the synthesis and activity of matrix metalloproteinases (MMPs), which degrade the joint matrix, are increased in the joint cells, thereby destroying the joint tissue.

In addition, arthritis worsens as the generation of nitric oxide (NO) by inflammatory cytokines and the subsequent production of self-amplifying cytokines induced by the generated nitric oxide lead to further synthesis of MMPs, thereby accelerating degradation of the joint matrix. At the same time, inflammatory cytokines increase the production of prostaglandin E2, a lipid metabolite, thereby inducing an inflammatory response in arthritis.

Plant-derived materials are excellent in terms of safety and have long been utilized. Particularly in Korea, the development of functional materials has been actively carried out mainly using plants and herbal ingredients that have been employed in folk remedies or traditional medicine.

However, the use of an extract of Potentilla chinensis or apigenin-7-O-glucuronide for the prevention, improvement, or treatment of arthritis has not been known, and studies on the underlying mechanism have not been conducted. Therefore, the inventors conducted direct efficacy studies on the prevention, improvement, or treatment of arthritis by the extract.

Accordingly, as a result of efforts to develop a composition for preventing, improving, or treating arthritis, the inventors confirmed that an extract of Potentilla chinensis or apigenin-7-O-glucuronide can prevent, improve, or treat arthritis, thereby completing the present invention.

### DISCLOSURE

### Technical Problem

Therefore, the present invention has been made in view of the above problems, and it is one object of the present invention to provide a healthy functional food composition for prevention or improvement of arthritis, the composition including an extract of Potentilla chinensis or apigenin-7-O-glucuronide represented by Formula 1 below, or a pharmaceutically acceptable salt thereof, as an active ingredient:

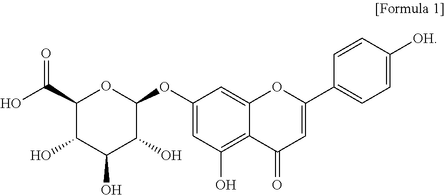 [SMILES: O=C(O)[C@H]1O[C@@H](Oc2cc(O)c3c(=O)cc(-c4ccc(O)cc4)oc3c2)[C@H](O)[C@@H](O)[C@@H]1O]

It is another object of the present invention to provide a pharmaceutical composition for the prevention or treatment of arthritis, including an extract of Potentilla chinensis, apigenin-7-O-glucuronide represented by Formula 1, or a pharmaceutically acceptable salt thereof, as an active ingredient.

It is still another object of the present invention to provide a quasi-drug composition for prevention or improvement of arthritis, including an extract of Potentilla chinensis, apigenin-7-O-glucuronide represented by Formula 1, or a pharmaceutically acceptable salt thereof, as an active ingredient.

It is still another object of the present invention to provide a topical composition for prevention or improvement of arthritis, including an extract of Potentilla chinensis, apigenin-7-O-glucuronide represented by Formula 1, or a pharmaceutically acceptable salt thereof, as an active ingredient.

It is still another object of the present invention to provide a method of preventing, improving, or treating arthritis, the method including administering an extract of Potentilla chinensis, apigenin-7-O-glucuronide represented by Formula 1, or a pharmaceutically acceptable salt thereof to a subject in need thereof.

It is yet another object of the present invention to provide use of an extract of Potentilla chinensis, apigenin-7-O-glucuronide represented by Formula 1 below, or a pharmaceutically acceptable salt thereof for the preparation of a composition for the prevention, improvement, or treatment of arthritis.

### Technical Solution

In accordance with an aspect of the present invention, the above and other objects can be accomplished by the provision of a healthy functional food composition for prevention or improvement of arthritis, including an extract of Potentilla chinensis, apigenin-7-O-glucuronide represented by Formula 1 below, or a pharmaceutically acceptable salt thereof, as an active ingredient.

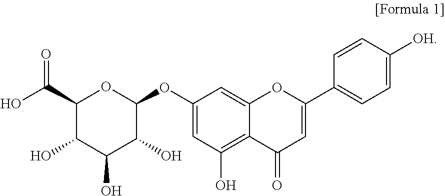 [SMILES: O=C(O)[C@H]1O[C@@H](Oc2cc(O)c3c(=O)cc(-c4ccc(O)cc4)oc3c2)[C@H](O)[C@@H](O)[C@@H]1O]

The Potentilla chinensis is a perennial herb of the family Asteraceae, and is a plant in which the leaf is designated as an edible part in the list of food ingredients notified by the Ministry of Food and Drug Safety.

The extract of Potentilla chinensis may be an extract obtained by extracting the whole aerial part of the plant, a part thereof, or a material derived therefrom, with a solvent. The part may be, for example, the stem, root, leaf, flower, petal, seed, pulp, fruit peel, or fruit of the plant, and is preferably the leaf. The whole, part, or material derived therefrom used for extraction may be pulverized, cut, or suitably dried.

The extract of Potentilla chinensis may be obtained by any extraction method used for extracting natural plants, such as hot-water extraction, solvent extraction, distillation extraction, or supercritical extraction, and is preferably extracted with water, an organic solvent, or a mixed solvent thereof. The organic solvent may be one or more selected from the group consisting of C1-4 alcohols, for example, ethanol, methanol, isopropanol, and butanol. The alcohol may include potable alcohol. The concentration of alcohol in an aqueous alcohol solution may be 1 to 99.5% (v/v), for example 10 to 99.5% (v/v), 1 to 70% (v/v), 1 to 40% (v/v), 5 to 50% (v/v), 5 to 40% (v/v), 10 to 50% (v/v), or 10 to 40% (v/v).

The extraction may include adding the extraction solvent in an amount of 3 to 50 parts by weight, for example 3 to 40 parts by weight, 3 to 30 parts by weight, 5 to 50 parts by weight, 5 to 40 parts by weight, 5 to 30 parts by weight, or 4 to 40 parts by weight, relative to Potentilla chinensis. For example, the extraction may include adding 3 to 50 kg of the solvent to 1 kg of a material derived from Potentilla chinensis.

The extraction may be carried out by methods such as hot-water extraction, immersion extraction, supercritical extraction, subcritical extraction, reflux cooling extraction, steam distillation, high-pressure enzymatic hydrolysis, ultrasonic extraction, leaching, or pressing.

The extraction may be carried out at 4° C. to 90° C., for example 4° C. to 80° C., 4° C. to 70° C., 5° C. to 80° C., 10° C. to 70° C., 15° C. to 70° C., or 20° C. to 70° C. The extraction time may vary depending on the selected temperature, and may be 1 hour to 2 months, for example 1 hour to 1 month, 1 hour to 10 days, 1 hour to 5 days, 1 hour to 3 days, 1 hour to 2 days, 1 hour to 1 day, 1 hour to 10 hours, 2 hours to 1 month, 2 hours to 15 days, 2 hours to 10 days, 2 hours to 5 days, 2 hours to 3 days, 2 hours to 2 days, 2 hours to 1 day, or 2 hours to 10 hours. The extraction may include mixing the whole of Potentilla chinensis, a part thereof, or a material derived therefrom with the solvent and leaving it for a certain time, which may include appropriate stirring. The extraction may be repeated one or more times, for example 1 to 5 times.

The extraction may further include separating plant residues and an extract solution by a known method such as filtration. The extraction may also include removing the solvent from the obtained extract solution by a known method such as vacuum concentration. The extraction may further include preparing a dry extract by drying the obtained extract using a method such as freeze-drying.

The apigenin-7-O-glucuronide may be isolated from an extract of Potentilla chinensis, and specifically may be a fraction of the extract of Potentilla chinensis. The term “fraction” refers to a substance separated from the extract containing a specific component. The apigenin-7-O-glucuronide may be isolated and purified using column chromatography. The chromatography may be selected from silica gel column chromatography, HP-20 column chromatography, RP-18 column chromatography, LH-20 column chromatography, high-performance liquid chromatography (HPLC), or a combination thereof.

In an embodiment, the extract of Potentilla chinensis or the apigenin-7-O-glucuronide may be included in an amount of 0.0001 to 90.0% by weight, for example 0.01 to 60% by weight, 0.01 to 40% by weight, 0.01 to 30% by weight, 0.01 to 20% by weight, 0.01 to 10% by weight, 0.01 to 5% by weight, 0.05 to 60% by weight, 0.05 to 40% by weight, 0.05 to 30% by weight, 0.05 to 20% by weight, 0.05 to 10% by weight, 0.05 to 5% by weight, 0.1 to 60% by weight, 0.1 to 40% by weight, 0.1 to 30% by weight, 0.1 to 20% by weight, 0.1 to 10% by weight, 0.1 to 5% by weight, 5 to 20% by weight, 5 to 18% by weight, 6 to 15% by weight, 8 to 15% by weight, or 7 to 10% by weight, based on the total weight of the composition. However, for long-term intake for health or hygiene purposes or for health regulation, the amount may be less than the above range, and since there is no problem in terms of safety, the active ingredient may also be used in an amount exceeding the above range.

The extract of Potentilla chinensis or apigenin-7-O-glucuronide may be used as it is or in combination with other foods or food ingredients, and may be appropriately used according to conventional methods.

In an embodiment, the extract of Potentilla chinensis or apigenin-7-O-glucuronide may inhibit the expression or activity of one or more selected from the group consisting of IL-1β and COX-2.

In an embodiment, the extract of Potentilla chinensis or apigenin-7-O-glucuronide may inhibit the generation of nitric oxide (NO).

The extract of Potentilla chinensis or apigenin-7-O-glucuronide may inhibit the expression or activity of inflammatory cytokines such as interleukin-1β (IL-1β) and cyclooxygenase-2 (COX-2), and suppress the generation of nitric oxide (NO), an inflammatory mediator, thereby preventing, improving, or treating arthritis.

In an embodiment, the extract of Potentilla chinensis or apigenin-7-O-glucuronide may inhibit the expression or activity of one or more selected from the group consisting of MMP-1 and MMP-9.

In an embodiment, the extract of Potentilla chinensis or apigenin-7-O-glucuronide may increase the expression or activity of procollagen type II.

The extract of Potentilla chinensis or apigenin-7-O-glucuronide may inhibit the expression or activity of matrix metalloproteinases (MMPs), enzymes that destroy cartilage matrix components, and may increase the expression or activity of procollagen type II present in articular cartilage, thereby preventing, improving, or treating arthritis.

In an embodiment, the arthritis may be one or more selected from the group consisting of osteoarthritis, degenerative arthritis, rheumatoid arthritis, suppurative arthritis, ankylosing spondylitis, juvenile idiopathic arthritis, and Still's disease.

In the present description, the term “prevention” collectively refers to delaying or preventing the onset or recurrence of a disease, disorder, or a symptom thereof, in whole or in part; preventing acquisition or reacquisition of the disease or disorder; or reducing the risk of acquiring the disease or disorder. The prevention refers to any act of suppressing or delaying the occurrence of arthritis or arthritis-related diseases, disorders, or symptoms by administration of the composition according to the present invention.

In the present description, the term “improvement” refers to any act of at least reducing parameters associated with a treated condition, for example, the degree of symptoms.

In the present description, the term “treatment” refers to any act of alleviating or beneficially altering a disease, disorder, or a symptom thereof.

In the present description, the term “healthy functional food” refers to a food prepared or processed by extracting, concentrating, purifying, or mixing a specific component as a raw material for the purpose of health supplementation, or a specific component contained in food materials, and refers to a food designed and processed to sufficiently exhibit bioregulatory functions in the body, such as bioprotection, regulation of biorhythm, and prevention and recovery of diseases. The healthy functional food composition may perform functions related to the prevention or improvement of arthritis.

The type of food is not particularly limited. Examples of foods to which the extract may be added include formulations selected from the group consisting of powders, granules, tablets, capsules, pills, gels, jellies, suspensions, emulsions, syrups, tea bags, infusions, chewing gums, candies, and health drinks, and include all health foods in the ordinary sense.

The health drink composition may further include, as additional components, various sweeteners, flavoring agents, or natural carbohydrates, as in conventional beverages. The sweetener may be a natural sweetener or a synthetic sweetener. The natural sweetener may be thaumatin or a stevia extract, and the synthetic sweetener may be saccharin or aspartame.

The natural carbohydrate may be one or more selected from the group consisting of monosaccharides, disaccharides, polysaccharides, xylitol, sorbitol, and erythritol. The monosaccharide may be glucose or fructose, the disaccharide may be maltose or sucrose, and the polysaccharide may be dextrin or cyclodextrin. The proportion of the natural carbohydrate may generally be about 0.01 to 10 g, for example, about 0.01 to 0.1 g, per 100 ml of the composition according to the present invention.

The healthy functional food may further include food-acceptable additives and may include suitable carriers conventionally used in the manufacture of healthy functional foods.

In addition, the composition of the present invention may include various nutrients, vitamins, electrolytes, flavoring agents, colorants, pectic acid and salts thereof, alginic acid and salts thereof, organic acids, protective colloid thickeners, pH regulators, stabilizers, preservatives, glycerin, alcohols, and carbonating agents used in carbonated beverages. Furthermore, the composition of the present invention may include pulp for the manufacture of natural fruit juice, fruit juice beverages, and vegetable beverages. These components may be used independently or in combination. The proportion of these additives is not particularly critical but is generally selected within the range of 0.01 to 0.1 parts by weight per 100 parts by weight of the composition according to the present invention.

In accordance with another aspect of the present invention, provided is a pharmaceutical composition for the prevention or treatment of arthritis, the pharmaceutical composition including an extract of Potentilla chinensis, apigenin-7-O-glucuronide represented by Formula 1 below, or a pharmaceutically acceptable salt thereof, as an active ingredient:

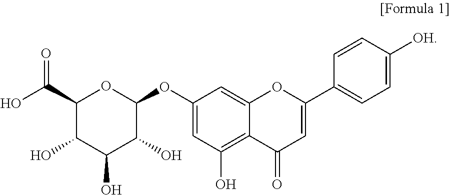 [SMILES: O=C(O)[C@H]1O[C@@H](Oc2cc(O)c3c(=O)cc(-c4ccc(O)cc4)oc3c2)[C@H](O)[C@@H](O)[C@@H]1O]

The effect of the extract of Potentilla chinensis or apigenin-7-O-glucuronide on arthritis is as described above.

In accordance with still another aspect of the present invention, provided is a method of preventing, improving, or treating arthritis, the method including administering an extract of Potentilla chinensis, apigenin-7-O-glucuronide represented by Formula 1 below, or a pharmaceutically acceptable salt thereof to a subject in need thereof:

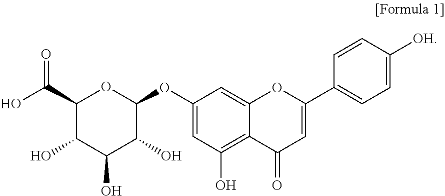 [SMILES: O=C(O)[C@H]1O[C@@H](Oc2cc(O)c3c(=O)cc(-c4ccc(O)cc4)oc3c2)[C@H](O)[C@@H](O)[C@@H]1O]

In accordance with still another aspect of the present invention, provided is use of an extract of Potentilla chinensis, apigenin-7-O-glucuronide represented by Formula 1 below, or a pharmaceutically acceptable salt thereof for the preparation of a composition for the prevention, improvement, or treatment of arthritis:

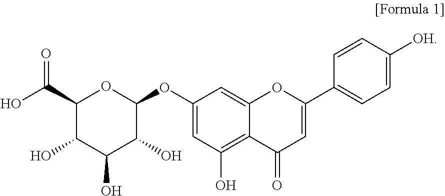 [SMILES: O=C(O)[C@H]1O[C@@H](Oc2cc(O)c3c(=O)cc(-c4ccc(O)cc4)oc3c2)[C@H](O)[C@@H](O)[C@@H]1O]

In an embodiment, the pharmaceutical composition may be formulated into a preparation selected from the group consisting of tablets, soft or hard capsules, pills, powders, suspensions, syrups, injections, and granules.

In an embodiment, the pharmaceutical composition may be for oral or parenteral administration.

The pharmaceutical composition may further include conventional fillers, extenders, binders, disintegrants, anti-caking agents, lubricants, wetting agents, pH regulators, nutrients, vitamins, electrolytes, alginic acid and salts thereof, pectic acid and salts thereof, protective colloids, glycerin, flavoring agents, emulsifiers, or preservatives.

The pharmaceutical composition may include pharmaceutically acceptable carriers, examples of which may include one or more selected from the group consisting of lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methylhydroxybenzoate, propyl hydroxybenzoate, talc, magnesium stearate, mineral oil, dextrin, calcium carbonate, propylene glycol, liquid paraffin, and saline.

The formulation of the pharmaceutical composition may vary depending on the method of use, and may be formulated by methods well known in the art to which the present invention pertains so as to provide rapid, sustained, or delayed release of the active ingredient after administration to a mammal.

Preparations for oral administration may include tablets, soft or hard capsules, pills, powders, suspensions, syrups, injections, and granules, and these preparations may be manufactured by mixing one or more excipients such as starch, calcium carbonate, sucrose, lactose, or gelatin. In addition to simple excipients, lubricants such as magnesium stearate or talc may also be used. Preparations for parenteral administration may include creams, lotions, ointments, liniments, solutions, aerosols, fluid extracts, elixirs, tinctures, sachets, patches, or injections.

The method may be applied to any animal, including humans and primates as well as livestock such as cattle, pigs, sheep, horses, dogs, and cats.

The dosage of the composition for the prevention, improvement, or treatment may be determined in consideration of the administration method, the age and sex of the subject, the severity and condition of the patient, the absorption and inactivation rate of the active ingredient in the body, and concomitant drugs. Based on the amount of active ingredient per day, the dosage may be 0.1 mg/kg to 500 mg/kg, 0.1 mg/kg to 400 mg/kg, or 1 mg/kg to 300 mg/kg of body weight, and may be administered once or in divided doses, but is not limited thereto.

In accordance with still another aspect of the present invention, provided is a quasi-drug composition for prevention or improvement of arthritis, including an extract of Potentilla chinensis, apigenin-7-O-glucuronide represented by Formula 1 below, or a pharmaceutically acceptable salt thereof, as an active ingredient:

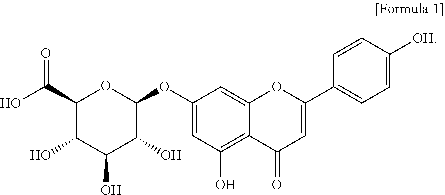 [SMILES: O=C(O)[C@H]1O[C@@H](Oc2cc(O)c3c(=O)cc(-c4ccc(O)cc4)oc3c2)[C@H](O)[C@@H](O)[C@@H]1O]

The effect of the extract of Potentilla chinensis or apigenin-7-O-glucuronide on arthritis is as described above

The term “quasi-drug” refers to an article used for the treatment, mitigation, handling, or prevention of diseases in humans or animals, which is not an instrument or machine and exerts weak or indirect effects on the human body, including articles used for sterilization, disinfection, or similar purposes to prevent infection. It refers to an article that is used for the diagnosis, treatment, mitigation, handling, or prevention of the condition or disease of humans or animals, excluding instruments, machines, or devices, and also refers to an article that is intended to exert a pharmacological effect on the structure or function of humans or animals but is not an instrument, machine, or device. Personal hygiene products may also be included.

When the extract is added to a quasi-drug composition, it may be used as it is or in combination with other quasi-drug ingredients, and may be appropriately used according to conventional methods. The mixing amount of the active ingredient may be suitably determined depending on the intended use (prevention, health, or therapeutic treatment).

The type or formulation of the quasi-drug composition of the present invention is not particularly limited, but may be, for example, bandages, gauze, absorbent cotton, plasters, disinfectants, cleansing agents, shower foams, gargles, wet tissues, detergent soaps, hand washes, humidifier fillers, masks, or filter fillers.

In accordance with yet another aspect of the present invention, provided is a topical composition for prevention or improvement of arthritis, including an extract of Potentilla chinensis, apigenin-7-O-glucuronide represented by Formula 1 below, or a pharmaceutically acceptable salt thereof, as an active ingredient:

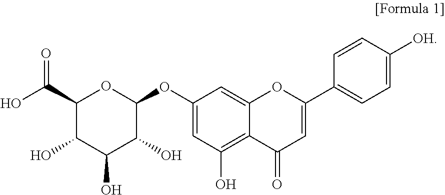 [SMILES: O=C(O)[C@H]1O[C@@H](Oc2cc(O)c3c(=O)cc(-c4ccc(O)cc4)oc3c2)[C@H](O)[C@@H](O)[C@@H]1O]

The effect of the extract of Potentilla chinensis or apigenin-7-O-glucuronide on arthritis is as described above.

The topical composition may be in the form of cream, gel, ointment, skin emulsion, skin suspension, transdermal patch, lotion, or a combination thereof. The topical composition may further include components commonly used in cosmetic or pharmaceutical external preparations, for example, aqueous ingredients, oily ingredients, powders, alcohols, moisturizers, thickeners, UV absorbers, whitening agents, preservatives, antioxidants, surfactants, fragrances, colorants, and various skin nutrients, alone or in appropriate combinations. The topical composition may also suitably contain metal chelators such as disodium edetate, trisodium edetate, sodium citrate, sodium polyphosphate, sodium metaphosphate, and gluconic acid; drugs such as caffeine, tannin, verapamil, licorice extract, glabridin, hot-water extracts of fruits such as quince, various herbal medicines, tocopherol acetate, glycyrrhetinic acid, tranexamic acid and derivatives or salts thereof, vitamin C, magnesium ascorbic phosphate, ascorbic glucoside, arbutin, kojic acid, glucose, fructose, and trehalose.

When the extract is added to the topical composition, it may be used as it is or in combination with other topical ingredients, and may be appropriately used according to conventional methods. The mixing amount of the active ingredient may be suitably determined depending on the intended use (prevention, health, or therapeutic treatment).

The skin includes all parts of the body skin, including the face, hands, arms, legs, feet, chest, abdomen, back, buttocks, and scalp.

### Advantageous Effects

A composition according to one aspect can inhibit the expression of MMP-1 and MMP-9, increase the expression of procollagen type II, and alleviate inflammation, thereby preventing, improving, or treating arthritis.

### DESCRIPTION OF DRAWINGS

FIG. 1 illustrates a ¹H-NMR spectrum (DMSO-d6, D2O) of apigenin-7-O-glucuronide.

FIG. 2 illustrates a ¹³C-NMR spectrum (DMSO-d6, D2O) of apigenin-7-O-glucuronide.

FIG. 3 illustrates an HPLC spectrum of an extract of Potentilla chinensis and apigenin-7-O-glucuronide.

FIGS. 4A and 4B illustrate graphs showing the amount of NO generated by LPS after treatment with the extract of Potentilla chinensis and apigenin-7-O-glucuronide.

FIGS. 5A and 5B illustrate the expression level of LPS-induced IL-1β mRNA after treatment with the extract of Potentilla chinensis and apigenin-7-O-glucuronide.

FIGS. 6A and 6B illustrate the expression level of LPS-induced COX-2 mRNA after treatment with the extract of Potentilla chinensis and apigenin-7-O-glucuronide.

FIGS. 7A and 7B illustrate the amount of PMA-induced MMP-1 secretion after treatment with the extract of Potentilla chinensis and apigenin-7-O-glucuronide.

FIG. 8 illustrates the amount of PMA-induced MMP-9 secretion after treatment with apigenin-7-O-glucuronide.

FIGS. 9A and 9B illustrate the amount of IL-1β-induced Collagen Type II Alpha 1 synthesized after treatment with the extract of Potentilla chinensis and apigenin-7-O-glucuronide.

FIGS. 10A and 10B illustrate carrageenan-induced time-dependent paw edema and the inhibitory effect of treatment with the extract of Potentilla chinensis.

FIGS. 11A and 11B illustrate pain response measurement (Randall-Selitto) and mechanical allodynia (Von-Frey) in animals with carrageenan-induced inflammatory pain, after treatment with the extract of Potentilla chinensis.

FIGS. 12A, 12B, and 12C illustrate the inhibitory effect on paw edema in an adjuvant-induced arthritis (AIA) animal model after treatment with the extract of Potentilla chinensis.

### BEST MODE

The present invention will now be described in more detail with reference to the following embodiments. However, these embodiments are provided for illustrative purposes only, and the scope of the present invention is not limited thereto.

The terms used in the present specification are used to explain a specific exemplary embodiment and not to limit the present inventive concept. Thus, the expression of singularity in the present specification includes the expression of plurality unless clearly specified otherwise in context. It will be further understood that the terms “comprise” and/or “comprising”, when used in this specification, specify the presence of stated components, steps, operations, and/or elements, but do not preclude the presence or addition of one or more other components, steps, operations, and/or elements thereof.

It should not be understood that arbitrary aspects or designs disclosed in “embodiments”, “examples”, “aspects”, etc. used in the specification are more satisfactory or advantageous than other aspects or designs.

In addition, the expression “or” means “inclusive or” rather than “exclusive or”. That is, unless otherwise mentioned or clearly inferred from context, the expression “x uses a or b” means any one of natural inclusive permutations.

In addition, as used in the description of the invention and the appended claims, the singular forms “a”, “an” and “the” are intended to include the plural forms as well, unless context clearly indicates otherwise.

In addition, when an element such as a layer, a film, a region, and a constituent is referred to as being “on” another element, the element can be directly on another element or an intervening element can be present

### Example 1. Preparation of Extract of Potentilla chinensis

To prepare an extract of Potentilla chinensis, a wild Potentilla chinensis plant was purchased in Korea, washed with distilled water, and completely dried in a cool and shaded place until no change in weight was observed.

The dried material was pulverized using a homogenizer, and 30 g of the pulverized material was added to 900 g of an extraction solvent prepared by mixing ethanol and distilled water at a weight ratio of 3:7. At this time, the mixing ratio of the pulverized material to the extraction solvent was set at 1:30 (w/w). The mixture was subjected to extraction in a shaking water bath at 80° C. for 5 hours. The extract obtained was filtered at room temperature through a Whatman No. 2 filter paper to remove insoluble materials.

Next, the extract was concentrated under reduced pressure using a distillation apparatus equipped with a cooling condenser to completely remove the solvent, thereby obtaining an extract of Potentilla chinensis.

### Example 2: Preparation of Apigenin-7-O-Glucuronide

A suspension was prepared by adding an equal weight of distilled water to the extract powder obtained in Example 1. The suspension was subjected to gel filtration using Diaion HP-20 resin. A developing solvent was a mixed solution of [distilled water:methanol=1:0~0:1], and the extract was fractionated into 13 subfractions (Fr. 1-13). Subfraction 6 was further subjected to gel filtration using Sephadex LH-20 resin. The developing solvent was a mixed solution of [methanol:distilled water=8:2], and the fraction was divided into 10 subfractions (Fr. 6-1-10). Among them, subfraction 6-5 was further subjected to gel filtration using silica gel resin. The developing solvent was a mixed solution of [methylene chloride:methanol:distilled water=7:3:0~0:0:1], and the fraction was separated into 3 subfractions (Fr. 6-5-1-5). From subfraction 6-5-3, A pure single compound of apigenin-7-O-glucuronide (Compound 1) was obtained.

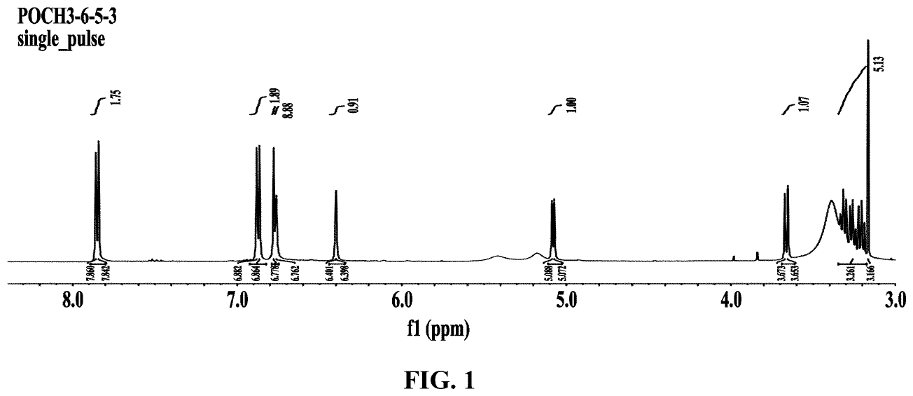

To confirm the structure of Compound 1, ¹H-NMR was analyzed (see FIG. 1). The results were as follows:

¹H NMR (DMSO-d6, 500 MHz): δH 7.85 (2H, d, J=9.0 Hz, H-2′,6′), 6.87 (2H, d, J=9.0 Hz, H-3″,5″), 6.77 (1H, m, H-6, 8), 6.40 (1H, s, H-3), 5.08 (1H, d, J=8.0 Hz, H-1″″), 3.66 (1H, d, J=10.0 Hz, H-5″), 3.26 (3H, m, H-2″″, 3″″, 4″″)

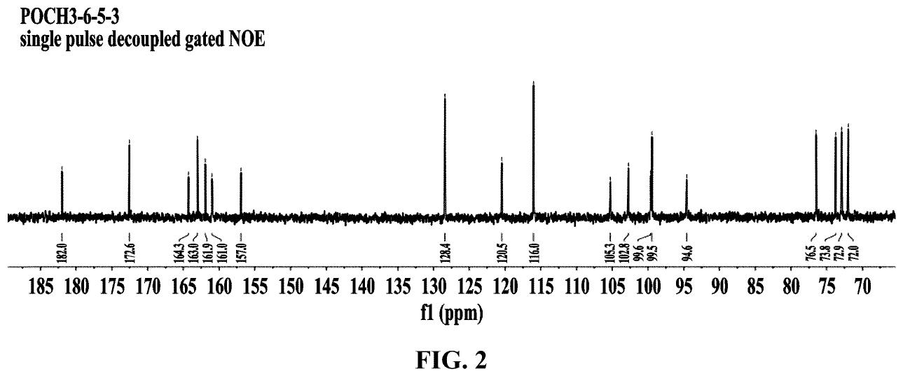

The ¹³C-NMR spectrum (see FIG. 2) was as follows:

¹³C NMR (DMSO-d6, 125 MHz) δC 182.0 (C-4), 172.6 (C-6″), 164.3 (C-2), 163.0 (C-7), 161.9 (C-5), 161.0 (C-4′), 157.0 (C-9), 128.4 (C-2′,6′), 120.5 (C-1′), 116.0 (C-3′,5′), 105.3 (C-10), 102.8 (C-3), 99.6 (C-1″), 99.5 (C-6), 94.6 (C-8), 73.8 (C-3″), 76.5 (C-5″), 72.9 (C-2″), 72.0 (C-4″).

Taken together with the above results and comparison with the literature (Baris et al., 2011; Guvenalp et al., 2009), Compound 1 was identified as apigenin-7-O-glucuronide.

### Experimental Example 1: Content Analysis of Apigenin-7-O-Glucuronide in Extract of Potentilla chinensis

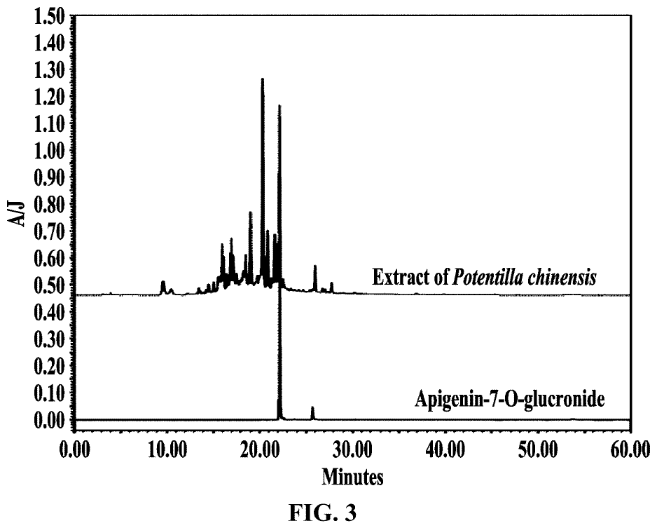

The extract powder obtained in Example 1 and apigenin-7-O-glucuronide were analyzed using high-performance liquid chromatography (HPLC) with a UV/Vis detector. HPLC was performed with a Waters e2695 Series system, a Waters 24489 UV/Vis detector (Worcester, MA, USA), and an X select C18 column (5 μm, 250×4.6 mm, Waters, Milford, MA, USA). All solvents used for the analysis were HPLC grade and purchased from J.T. Baker (Phillipsburg, NJ, USA). The column temperature was set at 35° C., the injection volume was 10 μL, and the detection wavelength was 325 nm. The mobile phase consisted of 0.1% formic acid in acetonitrile (ACN) and 0.1% formic acid in distilled water, and gradient analysis was performed for 50 minutes at a flow rate of 0.7 mL/min using a mixed solution of ACN and distilled water (0:10-10:0, v/v). For the test sample, 100 mg of the extract powder from Example 1 was precisely weighed, dissolved in 10 mL of methanol with ultrasonic treatment for 20 minutes, cooled to room temperature, and the supernatant was collected and filtered through a 0.45 μm membrane filter. Apigenin-7-O-glucuronide was precisely weighed at 1 mg, dissolved in 2 mL of methanol with ultrasonic treatment for 20 minutes, cooled to room temperature, further diluted with methanol, and filtered through a 0.45 μm membrane filter before analysis. Chromatograms of each sample were extracted at 325 nm, and the peaks of the extract of Potentilla chinensis and apigenin-7-O-glucuronide were compared and are shown in FIG. 3.

### Experimental Example 2. Confirmation of Inhibitory Effect of Potentilla chinensis Extract and Apigenin-7-O-Glucuronide on Nitric Oxide (NO) Production Induced by Lipopolysaccharide (LPS)

To confirm the NO-reducing effect of the extract of Potentilla chinensis from Example 1 and apigenin-7-O-glucuronide from Example 2, an inflammatory response was induced with LPS and subsequent changes in NO were measured.

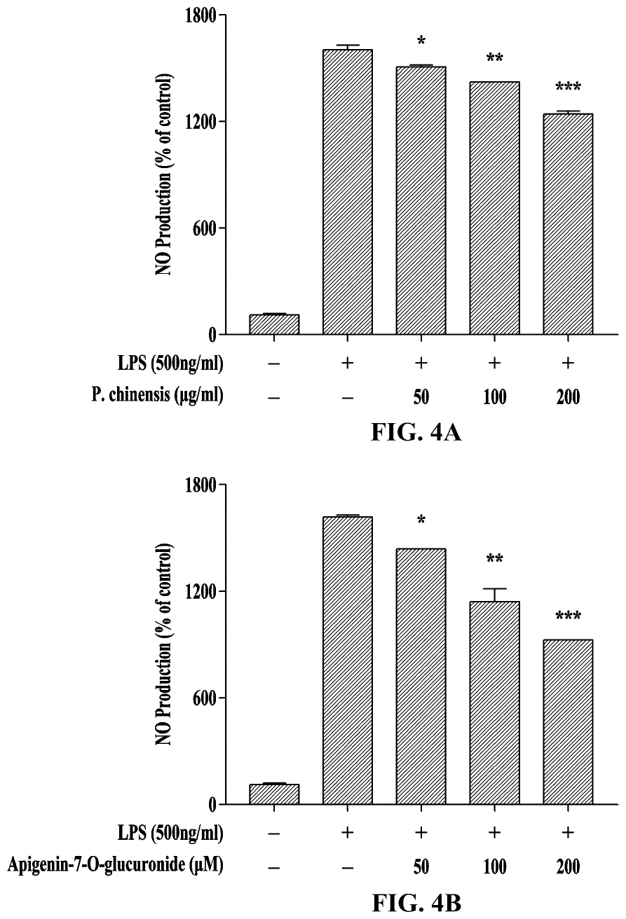

Specifically, RAW264.7 macrophages were obtained from the American Type Culture Collection (ATCC, MD, USA) and cultured in DMEM (Dulbecco's Modified Eagle's Medium) containing 10% FBS and 1% antibiotic-antimycotic under 5% CO₂ at 37° C. The cells were seeded into 6-well plates at a density of 8.0×10⁵ cells/1.5 mL/well and allowed to adhere for 24 hours, then treated with LPS (500 ng/ml) to induce inflammation. The extract of Potentilla chinensis from Example 1 was added at concentrations of 50, 100, and 200 ppm, and apigenin-7-O-glucuronide from Example 2 was added at concentrations of 50, 100, and 200 μM, followed by incubation for 24 hours. Next, 100 μL of the culture supernatant was transferred to a 96-well plate, mixed with 100 μL of Griess reagent (0.1% N-(1-naphtyl)ethylenediamine, 1% sulfanilamide), and reacted for 10 minutes. The absorbance of the resulting azo-derivative was measured at 540 nm using a microplate reader (BioTek, Vermont, USA). The measured absorbance was converted into nitrite concentration using a standard curve prepared with sodium nitrite dilutions, and the amount of NO produced was calculated. The results are shown in FIGS. 4A and 4B.

FIGS. 4A and 4B illustrate LPS-induced NO production after treatment with the extract of Potentilla chinensis and apigenin-7-O-glucuronide.

FIG. 4A illustrates NO production according to different concentrations of the extract of Potentilla chinensis, and FIG. 4B illustrates NO production according to different concentrations of apigenin-7-O-glucuronide.

As shown in FIGS. 4A and 4B, both the extract of Potentilla chinensis and apigenin-7-O-glucuronide significantly inhibited NO production in a concentration-dependent manner.

These results indicate that the extract of Potentilla chinensis and apigenin-7-O-glucuronide according to an embodiment can effectively inhibit NO production, thereby providing preventive, improving, or therapeutic effects on arthritis.

### Experimental Example 3. Confirmation of Inhibitory Effect of Potentilla chinensis Extract and Apigenin-7-O-Glucuronide on LPS-Induced IL-1β mRNA Expression

To confirm the inhibitory effect of the extract of Potentilla chinensis from Example 1 and apigenin-7-O-glucuronide from Example 2 on LPS-induced IL-1β mRNA expression, the expression level of IL-1β mRNA was analyzed using real-time polymerase chain reaction (PCR).

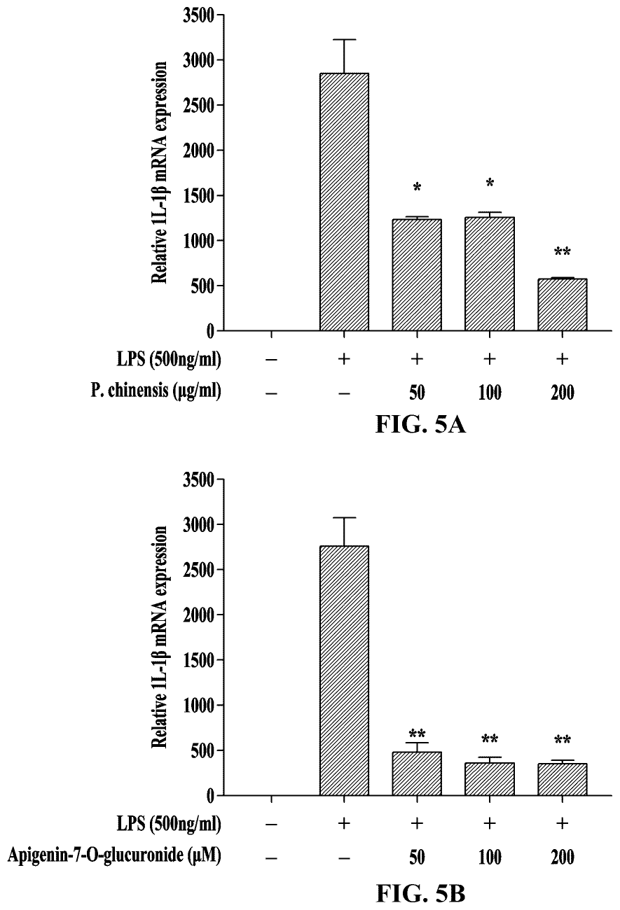

Specifically, RAW264.7 macrophages were obtained from the American Type Culture Collection (ATCC, MD, USA) and cultured in DMEM medium containing 10% FBS and 1% antibiotic-antimycotic under 5% CO2 at 37° C. The cells were seeded into 6-well plates at a density of 8.0×10⁵ cells/1.5 mL/well and allowed to adhere for 24 hours, then treated with LPS (500 ng/mL) in DMEM to induce inflammation. The extract of Potentilla chinensis from Example 1 was treated at concentrations of 50, 100, and 200 ppm, and apigenin-7-O-glucuronide from Example 2 was treated at concentrations of 50, 100, and 200 μM, followed by incubation for 24 hours. After 24 hours, IL-1β mRNA was extracted from the cells, synthesized into cDNA, and quantitative real-time PCR was performed using specific primers to evaluate the expression level of IL-1β. The results are shown in FIGS. 5A and 5B.

FIGS. 5A and 5B illustrate the expression level of LPS-induced IL-1β mRNA after treatment with the extract of Potentilla chinensis and apigenin-7-O-glucuronide.

FIG. 5A illustrates the expression level of IL-1β mRNA according to different concentrations of the extract of Potentilla chinensis, and FIG. 5B illustrates the expression level of IL-1β mRNA according to different concentrations of apigenin-7-O-glucuronide.

As shown in FIGS. 5A and 5B, both the extract of Potentilla chinensis and apigenin-7-O-glucuronide significantly inhibited IL-1β mRNA expression in a concentration-dependent manner.

These results indicate that the extract of Potentilla chinensis and apigenin-7-O-glucuronide according to an embodiment can effectively inhibit IL-1β mRNA expression, thereby providing preventive, improving, or therapeutic effects on arthritis.

### Experimental Example 4. Confirmation of Inhibitory Effect of Potentilla chinensis Extract and Apigenin-7-O-Glucuronide on LPS-Induced COX-2 mRNA Expression

To confirm the inhibitory efficacy of the extract of Potentilla chinensis from Example 1 and apigenin-7-O-glucuronide from Example 2 on LPS-induced COX-2 mRNA expression, the expression level of COX-2 mRNA was measured using real-time polymerase chain reaction (PCR).

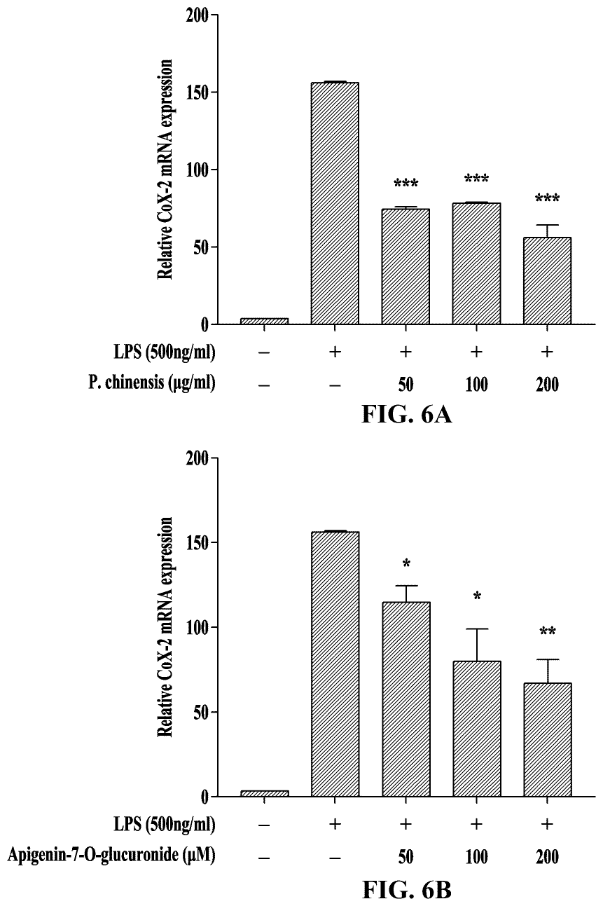

Specifically, RAW264.7 macrophages were obtained from the American Type Culture Collection (ATCC, MD, USA) and cultured in DMEM medium containing 10% FBS and 1% antibiotic-antimycotic under 5% CO2 at 37° C. The cells were seeded into 6-well plates at a density of 8.0×10⁵ cells/2.0 mL/well and allowed to adhere for 24 hours, then treated with LPS (500 ng/mL) in DMEM to induce inflammation. The extract of Potentilla chinensis from Example 1 was treated at concentrations of 50, 100, and 200 ppm, and apigenin-7-O-glucuronide from Example 2 was treated at concentrations of 50, 100, and 200 μM, followed by incubation for 24 hours. After 24 hours, COX-2 mRNA was extracted from the cells, synthesized into cDNA, and quantitative real-time PCR was performed using specific primers to evaluate the expression level of COX-2. The results are shown in FIGS. 6A and 6B.

FIGS. 6A and 6B illustrate the expression level of LPS-induced COX-2 mRNA after treatment with the extract of Potentilla chinensis and apigenin-7-O-glucuronide.

FIG. 6A illustrates the expression level of COX-2 mRNA according to different concentrations of the extract of Potentilla chinensis, and FIG. 6B illustrates the expression level of COX-2 mRNA according to different concentrations of apigenin-7-O-glucuronide.

As shown in FIGS. 6A and 6B, both the extract of Potentilla chinensis and apigenin-7-O-glucuronide significantly inhibited COX-2 mRNA expression.

These results indicate that the extract of Potentilla chinensis and apigenin-7-O-glucuronide according to an embodiment can effectively inhibit COX-2 mRNA expression, thereby providing preventive, improving, or therapeutic effects on arthritis.

### Experimental Example 5. Confirmation of Inhibitory Effect of Potentilla chinensis Extract and Apigenin-7-O-Glucuronide on PMA-Induced MMP-1 Expression in Chondrocytes

To evaluate the inhibitory efficacy of the extract of Potentilla chinensis from Example 1 and apigenin-7-O-glucuronide from Example 2 on PMA-induced MMP-1 production in chondrocytes, the amount of MMP-1 secreted was measured using a Human MMP-1 ELISA kit (ab100603, Abcam, USA).

Specifically, SW-1353 chondrocytes were obtained from the American Type Culture Collection (ATCC, MD, USA) and cultured in DMEM medium containing 10% FBS and 1% antibiotic-antimycotic under 5% CO₂ at 37° C. The cells were seeded into 6-well plates at a density of 2.0×10⁵ cells/2.0 mL/well and allowed to adhere for 24 hours, then treated with PMA (50 ng/mL) in DMEM. The extract of Potentilla chinensis from Example 1 was treated at concentrations of 50, 100, and 200 ppm, and apigenin-7-O-glucuronide from Example 2 was treated at concentrations of 50, 100, and 200 μM, followed by incubation for 24 hours. After 24 hours, the amount of MMP-1 in the supernatant was quantified using the ELISA kit, and the results are shown in FIGS. 6A and 6B.

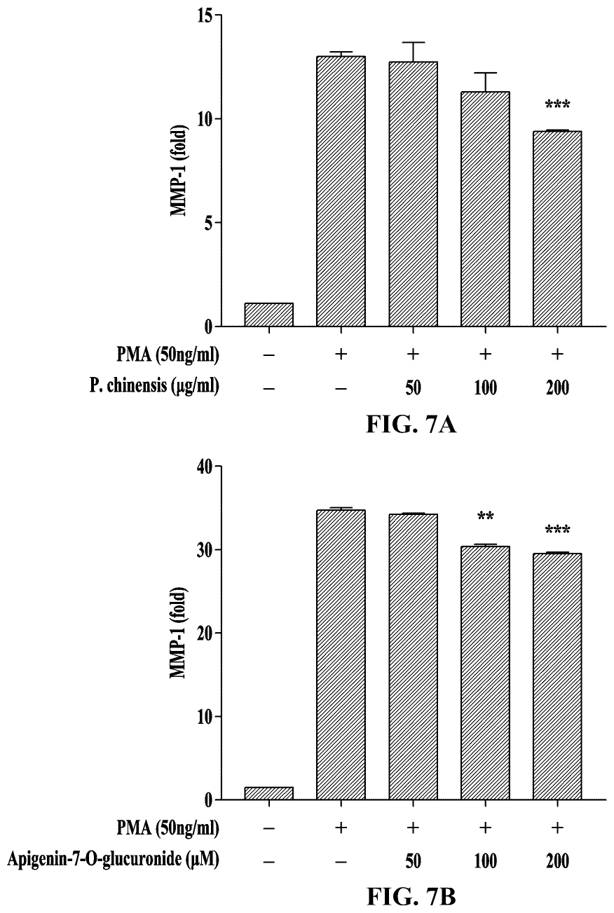

FIGS. 7A and 7B illustrate the amount of PMA-induced MMP-1 secretion after treatment with the extract of Potentilla chinensis and apigenin-7-O-glucuronide.

FIG. 7A illustrates the amount of MMP-1 secretion according to different concentrations of the extract of Potentilla chinensis, and FIG. 7B illustrates the amount of MMP-1 secretion according to different concentrations of apigenin-7-O-glucuronide.

As shown in FIGS. 7A and 7B, both the extract of Potentilla chinensis and apigenin-7-O-glucuronide significantly inhibited MMP-1 secretion in a concentration-dependent manner.

These results indicate that the extract of Potentilla chinensis and apigenin-7-O-glucuronide according to an embodiment can effectively inhibit MMP-1 secretion, thereby providing preventive, improving, or therapeutic effects on arthritis.

### Experimental Example 6. Confirmation of Inhibitory Effect of Apigenin-7-O-Glucuronide on PMA-Induced MMP-9 Expression in Chondrocytes

To evaluate the inhibitory efficacy of apigenin-7-O-glucuronide from Example 2 on PMA-induced MMP-9 production in chondrocytes, the amount of MMP-9 secreted was measured using a Human MMP-9 ELISA kit (ab246539, Abcam, USA).

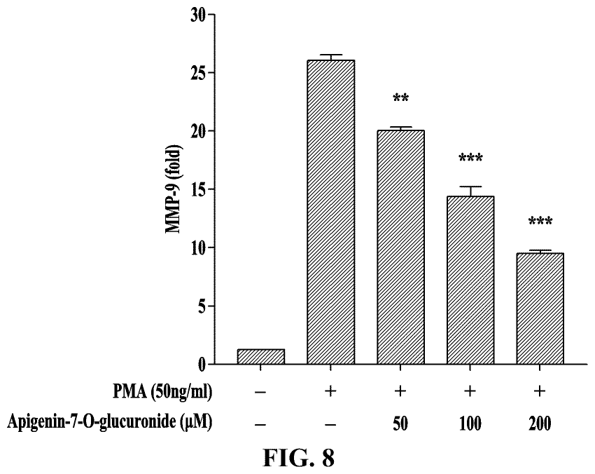

Specifically, SW-1353 chondrocytes were obtained from the American Type Culture Collection (ATCC, MD, USA) and cultured in DMEM medium containing 10% FBS and 1% antibiotic-antimycotic under 5% CO₂ at 37° C. The cells were seeded into 6-well plates at a density of 2.0×10⁵ cells/2.0 mL/well and allowed to adhere for 24 hours, then treated with PMA (50 ng/mL) in DMEM. The extract of Potentilla chinensis from Example 1 was treated at concentrations of 50, 100, and 200 ppm, and apigenin-7-O-glucuronide from Example 2 was treated at concentrations of 50, 100, and 200 μM, followed by incubation for 24 hours. After 24 hours, the amount of MMP-1 in the supernatant was quantified using the ELISA kit, and the results are shown in FIG. 8.

FIG. 8 illustrates the amount of PMA-induced MMP-9 secretion after treatment with apigenin-7-O-glucuronide.

As shown in FIG. 8, apigenin-7-O-glucuronide significantly inhibited MMP-9 secretion in a concentration-dependent manner.

These results indicate that the extract of Potentilla chinensis and apigenin-7-O-glucuronide according to an embodiment can effectively inhibit MMP-9 secretion, thereby providing preventive, improving, or therapeutic effects on arthritis.

### Experimental Example 7. Confirmation of Stimulatory Effect of Potentilla chinensis Extract and Apigenin-7-O-Glucuronide on IL-1β-Induced Collagen Type II Alpha 1 Synthesis In Chondrocytes

To evaluate the stimulatory efficacy of the extract of Potentilla chinensis from Example 1 and apigenin-7-O-glucuronide from Example 2 on IL-1β-induced Collagen Type II Alpha 1 synthesis in chondrocytes, the secretion of Collagen Type II Alpha 1 was measured using a Human Collagen II Alpha 1 ELISA kit (NBP2-75853, Novus, USA).

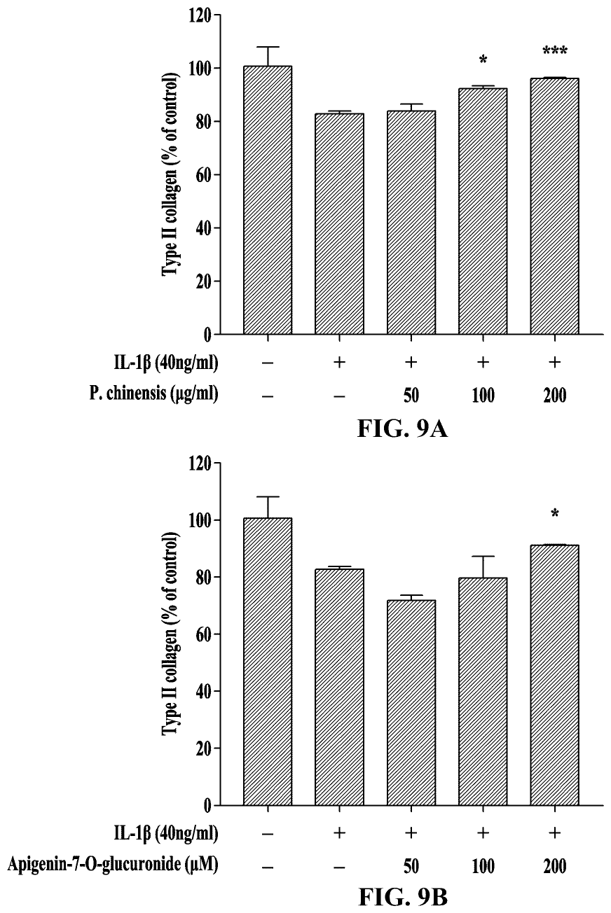

Specifically, SW-1353 chondrocytes were obtained from the American Type Culture Collection (ATCC, MD, USA) and cultured in DMEM medium containing 10% FBS and 1% antibiotic-antimycotic under 5% CO₂ at 37° C. The cells were seeded into 6-well plates at a density of 2.0×10⁵ cells/2.0 mL/well and allowed to adhere for 24 hours, then treated with IL-1β (40 ng/ml) in DMEM. The extract of Potentilla chinensis from Example 1 was treated at concentrations of 50, 100, and 200 ppm, and apigenin-7-O-glucuronide from Example 2 was treated at concentrations of 50, 100, and 200 μM, followed by incubation for 24 hours. After 24 hours, the amount of Collagen II Alpha 1 in the supernatant was quantified using the ELISA kit, and the results are shown in FIGS. 9A and 9B.

FIG. 9 illustrates the amount of IL-1β-induced Collagen Type II Alpha 1 synthesized after treatment with the extract of Potentilla chinensis and apigenin-7-O-glucuronide.

FIG. 9A illustrates the amount of Collagen Type II Alpha 1 synthesis according to different concentrations of the extract of Potentilla chinensis, and FIG. 9B illustrates the amount of Collagen Type II Alpha 1 synthesis according to different concentrations of apigenin-7-O-glucuronide.

As shown in FIGS. 9A and 9B, both the extract of Potentilla chinensis and apigenin-7-O-glucuronide significantly increased Collagen Type II Alpha 1 synthesis in a concentration-dependent manner.

### Experimental Example 8. Confirmation of Inhibitory Effect of Potentilla chinensis Extract on Carrageenan-Induced Paw Edema in Animals

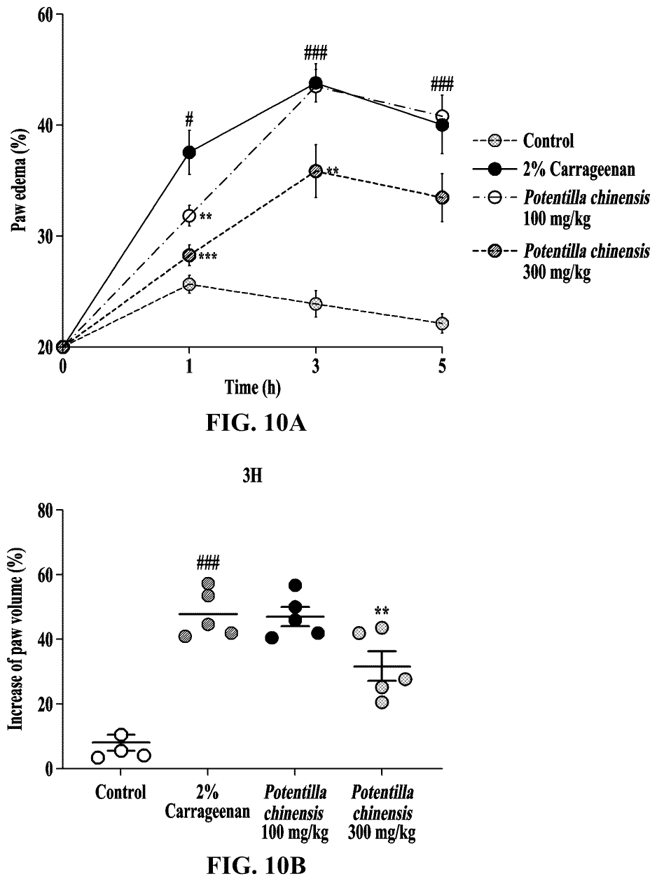

In Sprague Dawley rats (male, 200 g±10%), the paw volume of untreated animals was first measured using a plethysmometer. Sterile distilled water or the extract of Potentilla chinensis (100 mg/kg or 300 mg/kg, n=5 per group) was administered orally. Carrageenan (2%) was then injected subcutaneously into both hind paws, and paw volume changes were measured at 1, 3, and 5 hours after carrageenan injection using the plethysmometer (FIG. 10A). At 3 hours post-injection, inflammatory pain was measured after paw volume determination (FIG. 10B).

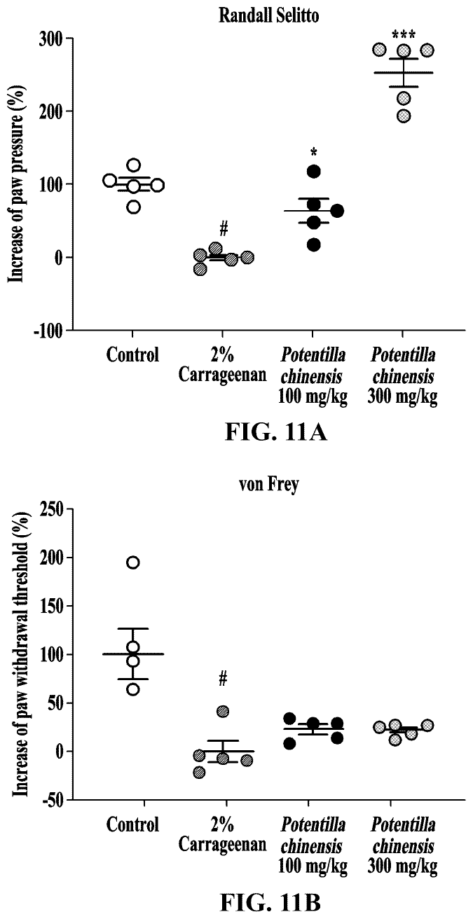

Using the Randall-Selitto test, suppression of inflammatory pain in the left paw was confirmed (FIG. 11A).

Using the Von-Frey test, suppression of mechanical allodynia in the right paw was confirmed (FIG. 11B).

These results indicate that the extract of Potentilla chinensis and apigenin-7-O-glucuronide according to an embodiment can effectively enhance Collagen Type II Alpha 1 synthesis and suppress inflammatory pain, thereby providing preventive, improving, or therapeutic effects on arthritis.

### Experimental Example 9. Confirmation of Inhibitory Effect of Potentilla chinensis Extract on Paw Edema in Adjuvant-Induced Arthritis (AIA) Animal Model

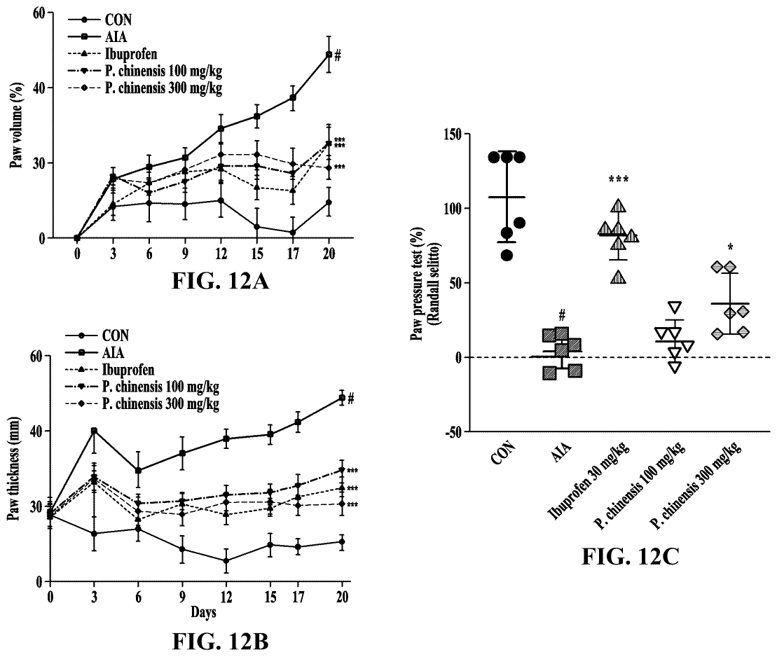

In Sprague Dawley rats (male, 200 g±10%), paw volumes of both hind paws were measured using a plethysmometer. Animals were orally administered sterile distilled water, ibuprofen (30 mg/kg), or the extract of Potentilla chinensis (100 mg/kg or 300 mg/kg, n=6 per group). Complete Freund's adjuvant (CFA) was injected into the left ankle joint, and volume changes in both paws were measured at intervals of approximately 3 days for 3 weeks using the plethysmometer (FIG. 12A). Paw thickness was measured using calipers (FIG. 12B). Pain response of the non-injected paw was measured using the Randall-Selitto test immediately before CFA injection and 20 days after CFA injection.

Using the Randall-Selitto test, suppression of inflammatory pain in the right paw was confirmed (FIG. 12C).

### Preparation Example 1. Preparation of Tablet

The extract of Potentilla chinensis from Example 1 and apigenin-7-O-glucuronide from Example 2 were mixed in the proportions shown in Table 1 below, and tablets were prepared by tableting according to a conventional tablet preparation method. The prepared tablets were confirmed to be stable under all test conditions of formulation examples.

TABLE 1

| | Unit | | Unit |
|---|---|---|---|
| Ingredient | weight (mg) | Ingredient | weight (mg) |
| Example 1 | 50 | Example 2 | 10 |
| Corn starch | 100 | Corn starch | 100 |
| Lactose | 100 | Lactose | 100 |
| Stearic acid | 2 | Stearic acid | 2 |

### Preparation Example 2. Preparation of Capsule

The extract of Potentilla chinensis from Example 1 and apigenin-7-O-glucuronide from Example 2 were mixed in the proportions shown in Table 2 below, filled into gelatin capsules, and soft capsules were prepared. The prepared capsules were confirmed to be stable under all test conditions of formulation examples.

TABLE 2

| | Unit | | Unit |
|---|---|---|---|
| Ingredient | weight (mg) | Ingredient | weight (mg) |
| Example 1 | 50 | Example 2 | 10 |
| Corn starch | 100 | Corn starch | 100 |
| Lactose | 100 | Lactose | 100 |
| Stearic acid | 2 | Stearic acid | 2 |

### Preparation Example 3. Preparation of Liquid Formulation

The extract of Potentilla chinensis from Example 1 and apigenin-7-O-glucuronide from Example 2 were mixed in the proportions shown in Table 3 below, and liquid formulations were prepared by filling into bottles or pouches according to a beverage preparation method suitable for preference. The prepared liquid formulations were confirmed to be stable under all test conditions of formulation examples.

TABLE 3

| | Unit | | Unit |
|---|---|---|---|
| Ingredient | weight (g) | Ingredient | weight (g) |
| Example 1 | 2.5050 | Example 2 | 0.5050 |
| Xanthan gum | 0.0075 | Xanthan gum | 0.0075 |
| Fructooligosaccharide | 0.7500 | Fructooligosaccharide | 0.7500 |
| solution | | solution | |
| Coconut flower | 1.0500 | Coconut flower | 1.0500 |
| extract powder | | extract powder | |
| Ssanghwa | 1.5000 | Ssanghwa | 1.5000 |
| concentrate | | concentrate | |
| Red ginseng flavor | 0.0450 | Red ginseng flavor | 0.0450 |
| Purified water | 9.1425 | Purified water | 11.1425 |

### Preparation Example 4. Preparation of Jelly

The extract of Potentilla chinensis from Example 1 and apigenin-7-O-glucuronide from Example 2 were mixed in the proportions shown in Table 4 below, and jellies were prepared by filling into sachets according to a jelly preparation method suitable for preference. The prepared jellies were confirmed to be stable under all test conditions of formulation examples.

TABLE 4

| | Unit | | Unit |
|---|---|---|---|
| | weight | | weight |
| Ingredient | (g) | Ingredient | (g) |
| Example 1 | 2.0000 | Example 2 | 0.5000 |
| Food gel | 0.3600 | Food gel | 0.3600 |
| Carrageenan | 0.0600 | Carrageenan | 0.0600 |
| Calcium lactate | 0.1000 | Calcium lactate | 0.1000 |
| Sodium citrate | 0.0600 | Sodium citrate | 0.0600 |
| Complex | 0.0200 | Complex | 0.0200 |
| Scutellaria extract | | Scutellaria extract | |
| Enzyme-treated | 0.0440 | Enzyme-treated | 0.0440 |
| stevia | | stevia | |
| Fructooligosaccharide | 5.0000 | Fructooligosaccharide | 5.0000 |
| solution | | solution | |
| Red grape | 2.4000 | Red grape | 2.4000 |
| concentrate | | concentrate | |
| Purified water | 13.9560 | Purified water | 15.4560 |

The compositions of Preparation Examples 1 to 4 were formulated by mixing generally suitable components as formulation examples, but the mixing ratios and raw materials may be arbitrarily modified if necessary. The extract of Potentilla chinensis and the components derived therefrom of the present invention were stable under all formulation test conditions, and thus no problems were observed in formulation stability.

Although the present invention has been described through limited examples and drawings, the present invention is not intended to be limited to the examples. Those skilled in the art will appreciate that various modifications, additions and substitutions are possible, without departing from the scope and spirit of the invention. Therefore, the scope of the present invention should not be limited to the described examples, but should be defined not only by the claims described below but also by equivalents of these claims.

1. A healthy functional food composition for prevention or improvement of arthritis, comprising: an extract of Potentilla chinensis; apigenin-7-O-glucuronide represented by Formula 1 below; or a pharmaceutically acceptable salt thereof, as an active ingredient:

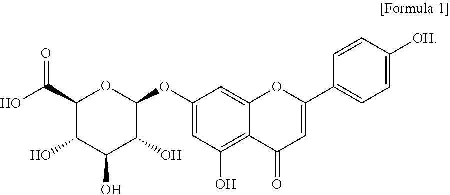 [SMILES: O=C(O)[C@H]1O[C@@H](Oc2cc(O)c3c(=O)cc(-c4ccc(O)cc4)oc3c2)[C@H](O)[C@@H](O)[C@@H]1O]

2. The healthy functional food composition according to claim 1, wherein the extract of Potentilla chinensis is extracted with water, a C1-C4 alcohol, or a mixed solvent thereof.

3. The healthy functional food composition according to claim 1, wherein the apigenin-7-O-glucuronide is isolated from the extract of Potentilla chinensis.

4. The healthy functional food composition according to claim 1, wherein the extract of Potentilla chinensis or the apigenin-7-O-glucuronide is comprised in an amount of 0.0001 to 90% by weight based on a total weight of the healthy functional food composition.

5. The healthy functional food composition according to claim 1, wherein the extract of Potentilla chinensis or the apigenin-7-O-glucuronide inhibits expression or activity of one or more selected from the group consisting of IL-1β and COX-2.

6. The healthy functional food composition according to claim 1, wherein the extract of Potentilla chinensis or the apigenin-7-O-glucuronide inhibits generation of nitric oxide (NO).

7. The healthy functional food composition according to claim 1, wherein the extract of Potentilla chinensis or the apigenin-7-O-glucuronide inhibits expression or activity of one or more selected from the group consisting of MMP-1 and MMP-9.

8. The healthy functional food composition according to claim 1, wherein the extract of Potentilla chinensis or the apigenin-7-O-glucuronide increases expression or activity of procollagen type II.

9. The healthy functional food composition according to claim 1, wherein the healthy functional food composition is formulated in a dosage form selected from the group consisting of powders, granules, tablets, capsules, pills, gels, jellies, suspensions, emulsions, syrups, tea bags, infusions, chewing gums, candies, and health drinks.

10. A pharmaceutical composition for prevention or treatment of arthritis, comprising: an extract of Potentilla chinensis; apigenin-7-O-glucuronide represented by Formula 1 below; or a food-acceptable or pharmaceutically acceptable salt thereof, as an active ingredient:

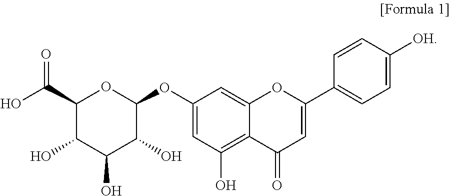 [SMILES: O=C(O)[C@H]1O[C@@H](Oc2cc(O)c3c(=O)cc(-c4ccc(O)cc4)oc3c2)[C@H](O)[C@@H](O)[C@@H]1O]

11. A quasi-drug composition for prevention or improvement of arthritis, comprising: an extract of Potentilla chinensis; apigenin-7-O-glucuronide represented by Formula 1 below; or a food-acceptable or pharmaceutically acceptable salt thereof, as an active ingredient:

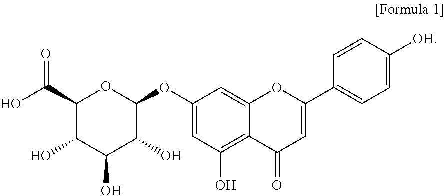 [SMILES: O=C(O)[C@H]1O[C@@H](Oc2cc(O)c3c(=O)cc(-c4ccc(O)cc4)oc3c2)[C@H](O)[C@@H](O)[C@@H]1O]

12. A topical composition for prevention or improvement of arthritis, comprising: an extract of Potentilla chinensis; apigenin-7-O-glucuronide represented by Formula 1 below; or a food-acceptable or pharmaceutically acceptable salt thereof, as an active ingredient: 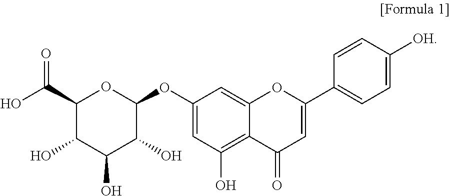 [SMILES: O=C(O)[C@H]1O[C@@H](Oc2cc(O)c3c(=O)cc(-c4ccc(O)cc4)oc3c2)[C@H](O)[C@@H](O)[C@@H]1O]
